(19) United States
(12) Patent Application Publication
(10) Pub. No.: US 2008/0269494 A1
(43) Pub. Date: Oct. 30, 2008

(54) Process for the Preparation of Alpha-Aryl-Alpha-Piperid-2-Yl-Acetamides and the Acid Hydrolysis Thereof

(76) Inventors: Marco Frigerio, Milano (IT); Sara Maculan, Gallarate (IT); Domenico Vergani, Biassono (IT)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/793,281

(22) PCT Filed: Dec. 16, 2005
(86) PCT No.: PCT/EP05/56862

§ 371 Date: Jan. 14, 2008

(30) Foreign Application Priority Data
Dec. 17, 2004 (IT) MI2004A002415

(51) Int. Cl. C07D 211/08

(52) U.S. Cl. 546/185

(57) ABSTRACT

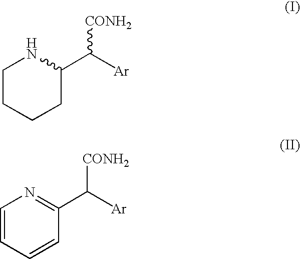

A process for the preparation of -aryl-piperid-2-yl-acetamides of formula (I) in which Ar is as defined in the disclosure, by catalytic reduction of α-aryl-α-pyridin-2-yl-acetamides (II) with rhodium catalysts. Acetamides of formula (II) can subsequently by hydrolysed to the corresponding arylacetic acids, e.g. ritalinic acid, a direct precursor of methylphenidate.

### FIELD OF THE INVENTION

The present invention relates to α-aryl-α-piperid-2-yl-acetamides, which are compounds useful for the preparation of arylacetic acids.

### TECHNOLOGICAL BACKGROUND

α-Aryl-α-piperid-2-yl-acetic acids (III)

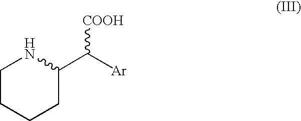

in which Ar is aryl
and the esters thereof are pharmaceutically useful compounds, mainly due to their effects on Central Nervous System. Methylphenidate (IV)

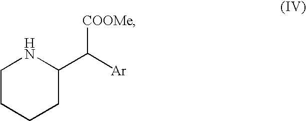

is, for example, a medicament used for the treatment of the hyperkinetic syndrome in children.

Acids (III) can be obtained by catalytic reduction of α-aryl-α-pyridinyl-2-yl-acetamides of formula (II)

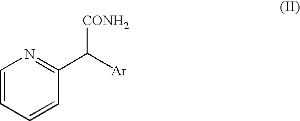

and subsequent hydrolysis of the resulting piperidylacetamide (I)

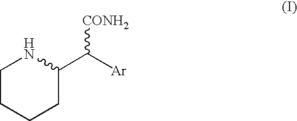

or by catalytic reduction of an α-aryl-α-α-pyrid-2-ylacetic acid salt or ester (V)

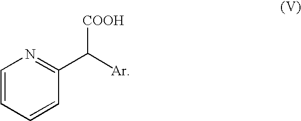

U.S. Pat. No. 2,838,519 and Journal of Labelled Compounds and Radiopharmaceuticals, vol. IX, No. 4, pp. 485-490 disclose e.g. the reduction of 2-phenyl-2-(2′-pyridyl)-acetamide by reduction with PtO₂ in glacial acetic acid, whereas the method described in J. Heterocyclic Chemistry involves the use of Pt/C.

Journal of Organic Chemistry 1962, vol. 27, pp. 284-286 describes the hydrogenation of pyridinecarboxylic acids with Rh/C as catalyst. According to the authors, this catalyst avoids the use of the acids usually necessary to prevent poisoning of the catalyst by the basic reaction substrate. The amount of catalyst is, however, high (40% on the pyridineacetic acid to reduce).

The use of catalysts based on Rh for the reduction of pyridineacetamides has not yet been disclosed.

### DISCLOSURE OF THE INVENTION

It has now been found that α-aryl-α-piperid-2-yl-acetamides of formula (I)

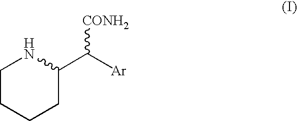

in which Ar is phenyl or naphthyl, optionally substituted with one or more C₁-C₃ alkyl groups, C₁-C₃ alkoxy groups, chlorine, fluorine, trifluoromethyl groups;
can be conveniently prepared by catalytic reduction of α-aryl-α-pyridin-2-yl-acetamides (II)

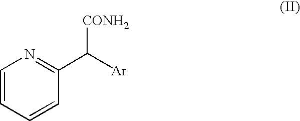

with a rhodium catalyst, preferably Rh/C, in a solvent which completely dissolves the α-aryl-α-pyridin-2-yl-acetamides and α-aryl-α-piperid-2-yl-acetamides, selected e.g. from acetic acid or a mineral acid aqueous solution, such as hydrochloric or sulfuric acid. The preferred solvent is acetic acid.

In the case of Rh/C, 1 g of catalyst is used per 10 g of compound of formula (II) (equivalent to 1 mmol of metal/193 mmoles of compound of formula II when Ar is phenyl), operating at a temperature ranging from 40 to 60° C., preferably from 50 to 55° C.

The process is particularly advantageous for the preparation of the amide (Ia)

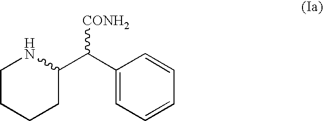

in which Ar is phenyl,
which amide is precursor of methylphenidate. In this case, the hydrogenation product is a d,l threo/erythro 10/90 mixture; after treatment with potassium hydroxide a d,l threo/erythro mixture higher than 70/30 is obtained which, by acid hydrolysis, yields d,l treo ritalinic acid (IIIa)

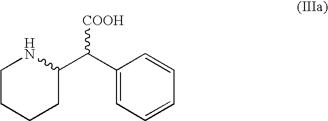

with purity higher than 99%.

The invention is illustrated in greater detail by the following example.

### EXAMPLE

### Preparation of Ritalinic Acid

### Step 1—Hydrogenation

A pressurized reactor is loaded with 20 g of 2-pyridyl-phenylacetamide and 70 ml of acetic acid, nitrogen is bubbled therein and 2 g of 5% Rh/C are added, and hydrogenation is carried out at 15 bas and 50-55° C. After approx. 5/6 hours, the catalyst is filtered off and the solution is concentrated under reduced pressure.

The residue is diluted with 20 ml of water and dripped into a potassium hydroxide solution at pH>11. The precipitated solid is filtered and used wet for the subsequent step.

### Step 2—Isomerization

The wet product from step 1 is suspended in 36 ml of water and added with 19.24 g of 90% potassium hydroxide. The obtained white suspension is heated at 95-105° C. for 6 hours. The mixture is then cooled to 0-5° C., filtered and washed with water. The resulting solid is dried under vacuum or used wet for the subsequent step.

### Step 3—Hydrolysis

A round-bottom 250 ml flask, fitted with magnetic stirrer, thermometer, condenser and dripping funnel, cooled with ice bath, is loaded with 20 g of the compound from step 2 suspended in 73 ml of water. 27 ml of 98% sulfuric acid are dropwise added to the suspension. The mixture is heated to 80-85° C. under stirring to complete hydrolysis of the amide (usually 8 hours), after that the solution is cooled to room temperature and poured in 350 ml of water. The solution is added with 1.2 g of carbon and left under stirring for 30 min., then filtered and washed with 30 ml of water. The pH of the solution is then adjusted to 6.0-6.2 with 30% NaOH. The resulting suspension is stirred at room temperature for 30 minutes, then filtered.

The resulting solid is washed with water and dried at 50° C. under vacuum overnight.

Yield: 10-15 g of ritalinic acid with purity above 99.0%.

1. A process for the preparation of α-aryl-α-piperid-2-yl-acetamides of formula (I) 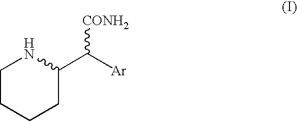
in which Ar is phenyl or naphthyl, optionally substituted with one or more C₁-C₃ alkyl groups, C₁-C₃ alkoxy groups, chlorine, fluorine, trifluoromethyl groups;
comprising the catalytic reduction of α-aryl-α-pyridin-2-yl-acetamides (II) 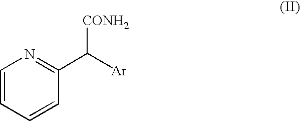
with a rhodium catalyst in a solvent which allows to completely dissolve the α-aryl-α-pyridin-2-yl-acetamides and the α-aryl-α-piperid-2-yl-acetamides.

2. The process as claimed in claim 1 wherein the solvent is selected from acetic acid or a hydrochloric or sulfuric acid aqueous solution.

3. The process as claimed in claim 2 in which the solvent is acetic acid.

4. The process according to claim 1 in which the catalyst is Rh/C.

5. The process as claimed in claim 4 in which Ig of catalyst per 10 grams of compound of formula (II) is used.

6. The process according to claim 1 in which the temperature ranges from 40 to 60° C.

7. The process according to claim 1 in which the temperature ranges from 50 to 55° C.

8. The process according to claim 1 in which Ar is phenyl.

9. The process for the preparation of ritalinic acid (Ilia) 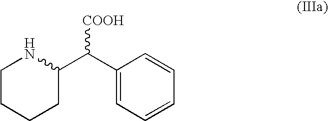
comprising the following steps:
a) preparation the amide (Ia) 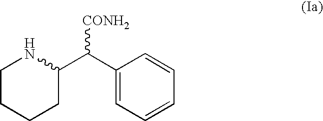
with the process of claim 8;
b) isomerization of the amide (Ia) to give a d,l mixture in which the threo/erythro ratio is higher than 70/30;
c) acid hydrolysis of the amide to give ritalinic acid.
